(19) United States
(12) Patent Application Publication
(10) Pub. No.: US 2003/0146081 A1
(43) Pub. Date: Aug. 7, 2003

(54) Acrylic acid recovery utilizing ethyl acrylate and selected co-solvents

(76) Inventors: Salvador Aldrett, Corpus Christi, TX (US); Diane Elizabeth Allen, Corpus Christi, TX (US); Nathan K. Powell, Pearl, TX (US); Christopher Lawrence Wilkins, Mathis, TX (US); Edmund Jungpiao Yang, Houston, TX (US)

Correspondence Address: M. Susan Spiering, c/o Celanese Ltd., P.O. Box 9077, Corpus Christi, TX, 78469-9077, US

(21) Appl. No.: 10/042,931

(22) Filed: Jan. 8, 2002

(51) Int. Cl. B01D 3/34; C07C 51/42; C07C 53/08

(52) U.S. Cl. 203/57; 203/2; 203/60; 203/43; 203/69; 203/70; 562/600; 562/607

(57) ABSTRACT

A method of recovering acrylic acid from a mixture comprising acrylic acid, water and acetic acid is disclosed, which includes: (a) extracting acrylic acid from the mixture with a solvent mixture comprising ethyl acrylate as the preponderant component thereof and an organic co-solvent selected from the group consisting of toluene, heptane, 1-heptene, methylcyclohexane, cycloheptane, cycloheptadiene, cycloheptatriene, 2,4-dimethyl-1,3 pentadiene, methylcyclohexene and methylenecyclohexene to form an extracted composition; and (b) azeotropically distilling the extracted composition to recover acrylic acid. Also disclosed is an alternate method of recovering acrylic acid which includes: (a) providing a feed stream containing acrylic acid, water, acetic acid, ethyl acrylate and an organic co-solvent selected from the group consisting of toluene, heptane, 1-heptene, methylcyclohexane, cycloheptane, cycloheptadiene, cycloheptatriene, 2,4-dimethyl-1,3 pentadiene, methylcyclohexene and methylenecyclohexene to a distillation column, wherein the weight ratio of ethyl acrylate to the organic co-solvent is from about 80:20 to about 95:5; and (b) azeotropically distilling said feed stream to provide an acrylic acid residue stream.

### TECHNICAL FIELD

The present invention relates to acrylic acid recovery from aqueous mixtures containing acrylic acid and impurities such as acetic acid, which mixtures may be obtained from the aqueous absorber of a conventional acrylic acid plant.

### BACKGROUND

Acrylic acid manufacture from propylene and acrolein is well known. Such processes are typically carried out in the gas phase and the gaseous reactor effluent is fed to the bottom of an aqueous absorber and cooled from a temperature of 250° C. or so to less than 80° C. by contact with aqueous acrylic acid. The water is fed to the top of the absorber at 30° C.-60° C., whereas the aqueous effluent from the absorber is then purified to recover acrylic acid. See Kirk-Othmer Encyclopedia of Chemical Technology, 3ed., Vol. 1, pp. 339-341 (Wiley, 1978).

Various methods have been employed to recover acrylic acid from the aqueous effluent. One method involves direct azeotropic distillation of the absorber effluent as described, for example, in U.S. Pat. No. 6,084,127 to Sakamoto et al. Another method of recovering acrylic acid from the aqueous mixture involves liquid-liquid extraction to extract acrylic acid into an organic phase followed by distillation of the organic phase to recover the acrylic acid. Regardless of the method employed, removal of close-boiling impurities, especially acetic acid, is problematical.

One method employed to remove impurities from acrylic acid is to employ direct azeotropic distillation as noted in the above '127 patent and yet another method employing azeotropic distillation is described in U.S. Pat. No. 3,433,831 of Yomiyama et al. In the method according to the '831 patent, acrylic acid is extracted from an aqueous mixture with an ethyl acrylate, organic co-solvent mixture and then the organic, acrylic acid containing composition is azeotropically distilled to recover the acrylic acid product.

The following additional references are believed illustrative of the art: U.S. Pat. No. 3,432,401 to Tcherkawski; U.S. Pat. No. 3,666,632 to Honda et al.; U.S. Pat. No. 3,859,175 to Ohrui et al.; U.S. Pat. No. 3,968,153 also to Ohrui et al.; U.S. Pat. No. 4,152,058 to Matsumura et al.; U.S. Pat. No. 4,166,774 to Wagner; U.S. Pat. No. 4,554,054 to Coyle; U.S. Pat. No. 5,154,800 to Berg; U.S. Pat. No. 5,315,037 to Sakamoto et al.; U.S. Pat. No. 5,759,358 to Bauer, Jr. et al.; U.S. Pat. No. 5,785,821 to Sakamoto et al.; U.S. Pat. No. 5,872,288 to Haramaki et al.; and U.S. Pat. No. 5,910,607 to Sakakura et al. See also, British Patent Specification No. 1,120,284 and Japanese Abstract JP 52153909.

### SUMMARY OF INVENTION

The present invention relates to the recovery of acetic acid from mixtures of acrylic acid, water and acetic acid such as those compositions obtained from the absorber of an acrylic acid unit manufacturing acrylic acid from propylene. There is thus provided in accordance with the present invention a method of recovering acrylic acid from a mixture comprising acrylic acid, water and acetic acid including: (a) extracting acrylic acid from the mixture with a solvent mixture comprising ethyl acrylate as the preponderant component thereof and an organic co-solvent selected from the group consisting of toluene, heptane, 1-heptene, methylcyclohexane, cycloheptane, cycloheptadiene, cycloheptatriene, 2,4-dimethyl-1,3 pentadiene, methylcyclohexene and methylenecyclohexene to form an extracted composition; and (b) azeotropically distilling the extracted composition to recover acrylic acid. Typically, the steps of extracting acrylic acid and azeotropically distilling the extracted composition are carried out in a continuous process to form a residue stream the preponderant component of which is acrylic acid. In many cases the residue stream is composed of at least 98% by weight acrylic acid and preferably the residue stream is composed of at least 99% acrylic acid. The residue stream typically contains less than about 0.75 wt % acetic acid, and preferably contains less than about 0.5 wt % acetic acid. So also, the residue stream typically contains less than about 0.5 wt % water and preferably the residue stream contains less than about 0.1 wt % water.

In most cases, the extracted composition comprises at least about 50 wt % ethyl acrylate and at least about 20 wt % acrylic acid.

A preferred organic co-solvent is toluene. The weight ratio of ethyl acrylate to the organic co-solvent in the solvent mixture is typically from about 80:20 to about 95:5 and preferably from about 85:15 to about 95:5.

Preferably, the foregoing process is operative to remove at least about 75 wt % of the acetic acid present in the mixture of acrylic acid, water and acetic acid undergoing purification, and more preferably, is operative to remove at least about 80 wt % of the acetic acid present in the mixture of acrylic acid, water and acetic acid undergoing purification.

In another aspect of the invention, there is provided a method of recovering acrylic acid including: (a) providing a feed stream containing acrylic acid, water, acetic acid, ethyl acrylate and an organic co-solvent selected from the group consisting of toluene, heptane, 1-heptene, methylcyclohexane, cycloheptane, cycloheptadiene, cycloheptatriene, 2,4-dimethyl-1,3 pentadiene, methylcyclohexene and methylenecyclohexene to a distillation column, wherein the weight ratio of ethyl acrylate to said organic co-solvent is from about 80:20 to about 95:5; and (b) azeotropically distilling the feed stream to provide an acrylic acid residue stream. The residue stream preferably contains at least about 98 wt % acrylic acid, and more preferably contains at least about 99 wt % acrylic acid. Generally, the feed stream may contain from about 5 to about 40 wt % water, from about 1 to about 4 wt % acetic acid and up to about 80 wt % acrylic acid. The residue stream, on the other hand, typically contains less than about 0.75 wt % acetic acid and, preferably the residue stream contains less than about 0.5 wt % acetic acid. Likewise, the residue stream usually contains less than about 0.5 wt % water. and more preferably, the residue stream contains less than about 0.1 wt % water. A preferred organic solvent is toluene and the weight ratio of ethyl acrylate to toluene in said feed stream is from about 85:15 to about 95:5 in a preferred embodiment.

Typically, the process is operative to remove at least about 75 wt % of the acetic acid present in the feed stream undergoing purification, and more preferably the distillation process is operative to remove at least about 80 wt % of the acetic acid present in said feed stream. Typical conditions include those wherein the azeotropic distillation is carried out with a temperature of about 100° C. about the lower portion of the distillation column and wherein the temperature about the central portion of the distillation column is maintained at a temperature of about 60° C. when azeotropically distilling the feed stream.

Still further aspects and advantages of the invention will become apparent from the discussion which follows.

### BRIEF DESCRIPTION OF DRAWING

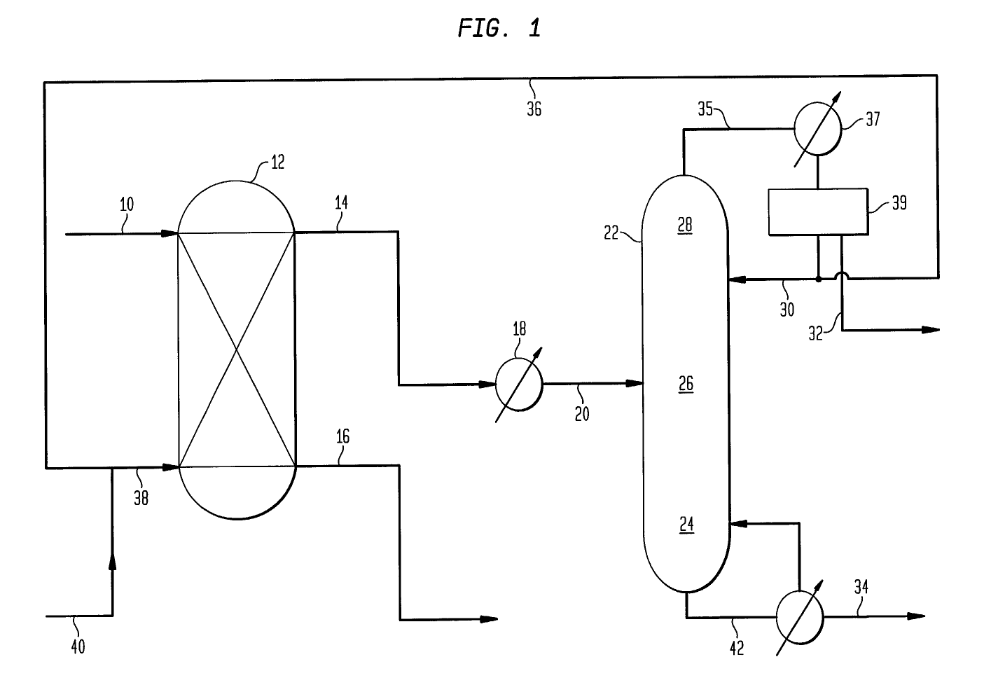

The invention is described in detail below with reference to FIG. 1 which is a schematic diagram illustrating the recovery of acrylic acid from an aqueous stream by way of extraction and azeotropic distillation.

### DETAILED DESCRIPTION

The present invention is exemplified and illustrated below for purposes of description only. Modifications within the spirit and scope of the present invention, set forth in the appended claims, will be readily apparent to those of skill in the art.

The following definitions are used herein:

Acrylic acid is sometimes referred to below as HAcA;

Acetic acid is sometimes referred to below as HOAc;

Ethyl acrylate is sometimes referred to below as EA or EtAcA;

Preponderant Component and the like refers to a component making up more than about fifty percent (50%) by weight of a mixture; and

Toluene is abbreviated “Tol” in some tables hereafter.

The term “Distribution Coefficient” and like terminology refers to the ratio of the weight percent (wt %) acrylic acid in the organic phase of an extraction performed to the wt % acrylic acid in the corresponding aqueous phase of the same extraction. A larger distribution coefficient for a given set of conditions thus generally indicates a more desirable extraction solvent.

The term “Selectivity” and like terminology as used herein refers to the ratio of the wt % of acrylic acid in the organic phase of an extraction performed to the wt % of water in the organic phase of that extraction. A larger Selectivity of a solvent thus means that solvent extracts acrylic acid more preferentially over water than a solvent with a lower Selectivity.

Unless otherwise specified or clear from the context: percent, ppm and the like refer to parts by weight.

### EXAMPLES

The invention will be better understood from the following examples. The values in the tables below are not normalized.

### Examples 1 and 2 and Comparative Example A

Determination of Distribution Coefficients

To a separatory funnel were added 50 g of a 34% by weight aqueous solution of acrylic acid and 50 g of a 90:10 by weight mixture of ethyl acrylate:toluene. The funnel was shaken vigorously for three minutes in order to extract the acrylic acid into the solvent, and the phases were allowed to separate. Each phase was analyzed for acrylic acid, water, and solvent content, and the distribution coefficient and selectivity for acrylic acid were determined. Distribution coefficients and selectivities using other ethyl acrylate:toluene compositions were established in a similar fashion. Table 1 below shows the results for the different solvents which were screened.

TABLE 1
Distribution Coefficients and Selectivities
for Mixed Solvents and Pure Ethyl Acrylate

| | Ethyl | | |
|---|---|---|---|
| | Acrylate:Toluene | Distribution | |
| Example | Ratio | Coefficient | Selectivity |
| 1 | 90:10 | 1.91 | 2.68 |
| 2 | 80:20 | 1.74 | 5.12 |
| A | 100:0 | 2.70 | 2.98 |

### Examples 3-8

Following the procedure of Examples 1-2 and Comparative Example A, extractions were carried out using mixed ethyl acrylate/toluene solvent systems. Results appear in Table 2 below.

TABLE 2
Extraction Using Ethyl Acrylate/Toluene Mixture In Various Proportions

| | | Wt. of 34% | | | Analytical Results | | | | | |
|---|---|---|---|---|---|---|---|---|---|---|
| | Type of | Acrylic Acid | Wt. Of | Sample | | % | | | Distribution | |
| Example | Sample | (g) | Solvent (g) | Wt. (g) | % EA | Toluene | % HACA | % H₂O | Coeff. | Selectivity |

| | | | | | | | | |
|---|---|---|---|---|---|---|---|---|
| — | 34% | x | x | x | x | x | 33.34 | 68.05 |
| | HACA | | | | | | | |

Extraction using 80/20 mixture of EA/Tol.

| | | | | | | | | | | |
|---|---|---|---|---|---|---|---|---|---|---|
| — | EA/Tol. | x | x | x | 80.38 | 22.29 | x | x | | |
| | Mix | | | | | | | | | |
| 3 | Aqueous | 50.0 | 50.0 | 29.1 | 2.06 | 0.01 | 10.23 | 88.85 | 1.7138 | 6.8364 |
| 3 | Organic | 50.0 | 50.0 | 67.5 | 63.53 | 17.64 | 17.53 | 2.56 | | |
| 4 | Aqueous | 50.0 | 50.0 | 33.2 | 2.09 | 0.01 | 10.16 | 87.07 | 1.7676 | 3.3948 |
| 4 | Organic | 50.0 | 50.0 | 66.5 | 62.02 | 16.85 | 17.96 | 5.29 | | |

Extraction using 90/10 mixture of EA/Tol.

| | | | | | | | | | | |
|---|---|---|---|---|---|---|---|---|---|---|
| — | EA/Tol. | x | x | x | 90.55 | 11.24 | x | x | | |
| | Mix | | | | | | | | | |
| 5 | Aqueous | 50.0 | 50.0 | 31.8 | 2.19 | 0.00 | 9.35 | 87.13 | 1.9018 | 2.7038 |
| 5 | Organic | 50.0 | 50.0 | 67.9 | 69.71 | 8.64 | 17.79 | 6.58 | | |
| 6 | Aqueous | 50.0 | 50.0 | 31.6 | 2.22 | 0.00 | 9.44 | 88.33 | 1.9175 | 2.6500 |
| 6 | Organic | 50.0 | 50.0 | 67.0 | 70.46 | 8.76 | 18.10 | 6.83 | | |

Extraction using 40:60 mixture of EA/Tol

| | | | | | | | | | | |
|---|---|---|---|---|---|---|---|---|---|---|
| 7 | Organic | 40.0 | 40.0 | 48.8 | 31.47 | 49.22 | 14.83 | 2.24 | 0.9693 | 6.6205 |
| 7 | Aqueous | 40.0 | 40.0 | 30.6 | 1.38 | 0.09 | 15.30 | 83.61 | | |

Extraction using 10:90 mixture of EA/Tol

| | | | | | | | | | | |
|---|---|---|---|---|---|---|---|---|---|---|
| 8 | Organic | 40.0 | 40.1 | 46.6 | 8.19 | 76.58 | 11.99 | 0.72 | 0.6022 | 16.6528 |
| 8 | Aqueous | 40.0 | 40.1 | 32.8 | 0.47 | 0.23 | 19.91 | 76.78 | | |

### Comparative Examples B, C, D

Following generally the procedure of Examples 3-8 above, the Distribution Coefficient and Selectivity of ethyl acrylate alone as an extraction solvent was evaluated as set forth in Table 3.

TABLE 3
Extraction Using Ethyl Acrylate as Solvent

| | | Wt. Of 34% | Wt. Of | | | | | | | |
|---|---|---|---|---|---|---|---|---|---|---|
| | Type of | Acrylic Acid | Solvent | | Sample | Analytical Results | | | Distribution | |
| Example | Sample | (g) | (g) | Temp. | Wt. (g) | % H₂O | % HAcA | % EA | Coeff. | Selectivity |
| B | Aqueous | 50.0 | 50.0 | 24.2 | 33.3 | 89.00 | 8.78 | 2.27 | 2.8018 | 3.0483 |
| B | Organic | 50.0 | 50.0 | | 66.5 | 8.07 | 24.60 | 70.20 | | |
| C | Aqueous | 50.0 | 50.0 | 24.3 | 29.8 | 88.80 | 8.98 | 2.24 | 2.7840 | 3.0414 |
| C | Organic | 50.0 | 50.0 | | 69.2 | 8.22 | 25.00 | 70.60 | | |
| D | Aqueous | 50.0 | 50.0 | 24.3 | 31.8 | 88.50 | 9.20 | 2.31 | 2.5217 | 2.8431 |
| D | Organic | 50.0 | 50.0 | | 68.0 | 8.16 | 23.20 | 72.70 | | |

### Comparative Examples E-P

Following generally the procedure of Comparative Examples B, C and D, toluene alone was evaluated as an extraction solvent for extracting acrylic acid from water. Details and results appear in Table 4 below.

TABLE 4
Extraction of Acrylic Acid with Toluene

| | | Wt. Of | | | | | | | | |
|---|---|---|---|---|---|---|---|---|---|---|
| | Type of | Acrylic Acid | Wt. Of | Wt. Of | Sample | Analytical Results | | | Distribution | |
| Example | Sample | (g) | H₂O (g) | Tol. (g) | Wt. (g) | % Toluene | % HACA | % H₂O | Coeff. | Selectivity |
| E | Aqueous | 2.0 | 48.0 | 50.0 | 50.0 | 0.08 | 3.41 | 95.40 | 0.1762 | 7.566204 |
| E | Organic | 2.0 | 48.0 | 50.0 | 49.9 | 95.98 | 0.60 | 0.08 | | |
| F | Aqueous | 2.0 | 48.0 | 50.0 | 50.0 | 0.09 | 3.24 | 92.71 | 0.1677 | 8.343558 |
| F | Organic | 2.0 | 48.0 | 50.0 | 49.6 | 97.46 | 0.54 | 0.07 | | |
| G | Aqueous | 13.0 | 45.0 | 42.0 | 53.7 | 0.19 | 15.93 | 82.38 | 0.4675 | 29.86367 |
| G | Organic | 13.0 | 45.0 | 42.0 | 46.0 | 90.04 | 7.45 | 0.25 | | |
| H | Aqueous | 13.0 | 45.0 | 42.0 | 53.9 | 0.18 | 15.71 | 82.67 | 0.4627 | 35.39698 |
| H | Organic | 13.0 | 45.0 | 42.0 | 45.9 | 88.33 | 7.27 | 0.21 | | |
| I | Aqueous | 20.0 | 42.0 | 38.0 | 55.4 | 0.37 | 23.81 | 72.40 | 0.5087 | 35.65665 |
| I | Organic | 20.0 | 42.0 | 38.0 | 44.2 | 84.23 | 12.11 | 0.34 | | |
| J | Aqueous | 20.0 | 42.0 | 38.0 | 55.6 | 0.37 | 23.67 | 75.61 | 0.5080 | 36.88037 |
| J | Organic | 20.0 | 42.0 | 38.0 | 44.2 | 83.61 | 12.02 | 0.33 | | |
| K | Aqueous | 26.0 | 39.0 | 35.0 | 57.1 | 0.57 | 31.12 | 67.08 | 0.4926 | 31.5111 |
| K | Organic | 26.0 | 39.0 | 35.0 | 42.6 | 80.03 | 15.33 | 0.49 | | |
| L | Aqueous | 26.0 | 39.1 | 35.0 | 57.1 | 0.66 | 30.66 | 69.20 | 0.4993 | 33.1838 |
| L | Organic | 26.0 | 39.1 | 35.0 | 42.6 | 80.33 | 15.31 | 0.46 | | |
| M | Aqueous | 34.0 | 35.0 | 31.0 | 60.4 | 1.73 | 41.00 | 57.39 | 0.4719 | 27.1060 |
| M | Organic | 34.0 | 35.0 | 31.0 | 39.5 | 76.03 | 19.35 | 0.71 | | |
| N | Aqueous | 34.0 | 35.0 | 31.0 | 60.3 | 1.82 | 40.86 | 56.17 | 0.4728 | 28.1439 |
| N | Organic | 34.0 | 35.0 | 31.0 | 39.6 | 76.31 | 19.32 | 0.69 | | |
| O | Aqueous | 41.0 | 32.0 | 27.0 | 64.4 | 3.38 | 48.29 | 48.87 | 0.4789 | 23.7863 |
| O | Organic | 41.0 | 32.0 | 27.0 | 35.5 | 72.29 | 23.13 | 0.97 | | |
| P | Aqueous | 41.0 | 32.0 | 27.0 | 64.3 | 3.36 | 48.07 | 48.32 | 0.4755 | 23.4249 |
| P | Organic | 41.0 | 32.0 | 27.0 | 35.4 | 71.83 | 22.86 | 0.98 | | |

### Examples 9-20 and Comparative Example Q

Following generally the procedure of the above examples, additional solvent compositions were evaluated as set forth in Table 5.

TABLE 5
Extraction with Miscellaneous Compositions

| | Type of | Wt. Acrylic | Wt. Of | Wt. Of | Wt. Of | Sample | Analytical Results | | | | Distribution | |
|---|---|---|---|---|---|---|---|---|---|---|---|---|
| Example | Sample | Acid (g) | H₂O (g) | EA. (g) | Tol. (g) | Wt. (g) | % EA | % Toluene | % HACA | % H₂O | Coeff. | Selectivity |
| 9 | Aqueous | 2.1 | 48.0 | 45.0 | 5.0 | 48.9 | 1.96 | 0.00 | 1.40 | 93.31 | 1.4707 | 1.3743 |
| 9 | Organic | 2.1 | 48.0 | 45.0 | 5.0 | 50.9 | 87.05 | 10.54 | 2.06 | 1.50 | | |
| 10 | Aqueous | 2.0 | 48.0 | 45.0 | 5.0 | 48.8 | 1.79 | 0.00 | 1.55 | 92.76 | 1.4736 | 1.1688 |
| 10 | Organic | 2.0 | 48.0 | 45.0 | 5.0 | 50.9 | 87.29 | 10.47 | 2.29 | 1.96 | | |
| 11 | Aqueous | 13.0 | 45.0 | 37.8 | 4.2 | 46.6 | 2.05 | 0.00 | 7.79 | 89.75 | 1.8860 | 2.9702 |
| 11 | Organic | 13.0 | 45.0 | 37.8 | 4.2 | 53.0 | 72.33 | 8.74 | 14.69 | 4.95 | | |
| 12 | Aqueous | 13.0 | 45.0 | 37.8 | 4.2 | 46.6 | 1.81 | 0.00 | 7.85 | 89.04 | 1.8939 | 2.5020 |
| 12 | Organic | 13.0 | 45.0 | 37.8 | 4.2 | 53.0 | 73.88 | 9.23 | 14.86 | 5.94 | | |
| 13 | Aqueous | 20.0 | 42.0 | 34.2 | 3.8 | 43.0 | 1.90 | 0.00 | 11.86 | 80.81 | 1.8967 | 2.9126 |
| 13 | Organic | 20.0 | 42.0 | 34.2 | 3.8 | 56.5 | 62.83 | 7.64 | 22.50 | 7.72 | | |
| 14 | Aqueous | 20.0 | 42.0 | 34.2 | 3.8 | 43.1 | 2.17 | 0.00 | 11.91 | 83.50 | 1.9286 | 2.5591 |
| 14 | Organic | 20.0 | 42.0 | 34.2 | 3.8 | 56.5 | 64.22 | 7.66 | 22.97 | 8.98 | | |
| 15 | Aqueous | 26.0 | 39.0 | 31.5 | 3.5 | 38.5 | 3.00 | 0.02 | 15.81 | 82.25 | 1.8640 | 2.9225 |
| 15 | Organic | 26.0 | 39.0 | 31.5 | 3.5 | 59.1 | 55.29 | 6.40 | 29.47 | 10.08 | | |
| 16 | Aqueous | 26.0 | 39.0 | 31.5 | 3.5 | 33.8 | 2.93 | 0.02 | 15.95 | 80.28 | 1.8783 | 2.3667 |
| 16 | Organic | 26.0 | 39.0 | 31.5 | 3.5 | 47.8 | 55.29 | 6.22 | 29.96 | 12.66 | | |
| 17 | Aqueous | 34.0 | 35.0 | 27.9 | 3.1 | 30.3 | 5.20 | 0.13 | 22.21 | 72.85 | 1.6418 | 1.9309 |
| 17 | Organic | 34.0 | 35.0 | 27.9 | 3.1 | 69.0 | 43.06 | 5.01 | 36.46 | 18.88 | | |
| 18 | Aqueous | 34.0 | 35.0 | 27.9 | 3.1 | 30.1 | 4.90 | 0.07 | 22.26 | 72.59 | 1.6512 | 1.9482 |
| 18 | Organic | 34.0 | 35.0 | 27.9 | 3.1 | 69.0 | 43.38 | 4.84 | 36.75 | 18.86 | | |
| 19 | Aqueous | 41.0 | 32.0 | 24.3 | 2.7 | 3.2 | 5.89 | 0.01 | 33.99 | 58.84 | 1.1937 | 1.3417 |
| 19 | Organic | 41.0 | 32.0 | 24.3 | 2.7 | 96.4 | 28.91 | 3.08 | 40.57 | 30.24 | | |
| 20 | Aqueous | 41.0 | 32.0 | 24.3 | 2.7 | 2.8 | 2.31 | 0.04 | 34.29 | 57.32 | 1.1594 | 1.2667 |
| 20 | Organic | 41.0 | 32.0 | 24.3 | 2.7 | 97.1 | 29.34 | 3.27 | 39.76 | 31.39 | | |
| Q | Aqueous | 46.0 | 33.0 | 0.0 | 21.0 | 73.4 | 0.00 | 4.28 | 51.18 | 44.05 | 0.4905 | 22.5415 |
| Q | Organic | 46.0 | 33.0 | 0.0 | 21.0 | 26.5 | 0.00 | 70.35 | 25.10 | 1.11 | | |

### Example 21 and Comparative Example R

A mixed solvent system approximately 90:10 ethyl acrylate:toluene was evaluated in an extraction/distillation purification system as shown in FIG. 1.

Referring to FIG. 1, an aqueous acrylic acid stream 10 is fed to a metal-packed extraction column 12. Stream 10 is typically slightly more than 60% water, about 35 percent acrylic acid and 2-3 percent acetic acid; that is, having the composition received from an aqueous absorber in a process for making acrylic acid from propylene as is known in the art.

Extractor 12 has an organic stream output 14 as well as an aqueous raffinate output 16. Raffinate stream 16 typically includes more than 90 percent water and may be further processed if so desired as is likewise known in the art. Stream 14 typically containing more than 25% of the desired acrylic acid product also typically contains about 50 to about 60 wt % ethyl acrylate solvent as well as acetic acid and water impurities.

Stream 14 is heated to 45-50° C. at 18 and is fed at 20 to a central portion 26 of a distillation column 22 as shown. In column 22 the stream fed at 20 is distilled with the following typical temperatures: at lower portion 24, the temperature is maintained at about 100° C.; at central portion 26, the temperature is maintained at about 60° C. and at upper portion 28, the temperature is maintained at slightly less than about 50° C. Reflux is supplied at 30; while an overhead stream 35 is cooled at 37, decanted at 39 to provide an organic solvent recycle stream 36 which is provided to extractor 12 at 38. An aqueous stream at 32 may be recycled or discarded. Make-up solvent is provided at 40.

The distillation residue exits column 22 at 42, is cooled to provide a product stream 34.

The foregoing apparatus was utilized to compare performance of an ethyl acrylate solvent system with a mixed ethyl acrylate/toluene solvent system as detailed below in Examples 21 and Comparative Example R. The compositions of the various streams are set forth in Table 6, whereas mass balances are given in Tables 7 and 8.

### Example 21

An aqueous stream composed of 34.99% by weight acrylic acid, 2.5% by weight acetic acid, and 62.44% by weight water is fed to the top of a counter-current extractor at a rate of 5.2 g/min and contacted with a solvent composed of 1.9% by weight acrylic acid, 1.38% by weight acetic acid, 85.33% by weight ethyl acrylate, 2.1% by weight water, and 9.29% by weight toluene, entering at the bottom of the extractor at a rate of 3.98 g/min. The extraction was performed with approximately 6 theoretical stages. The aqueous raffinate contained 2.5% by weight acrylic acid, 2.6% by weight acetic acid, 1.9% by weight ethyl acrylate, 92.99% by weight water, and 0.004% by weight toluene. The organic extract, composed of 27.38% by weight acrylic acid, 1.6% by weight acetic acid, 54.08% by weight ethyl acrylate, 10.7% by weight water, and 6.2% by weight toluene, was fed to a 20-tray one inch diameter Oldershaw distillation column at a rate of 6.2 g/min. The pressure at the top of the column was maintained at 165 mm Hg, the reflux rate at 2.8 ml/min, and the bottom temperature at 102° C. The condensed overhead was allowed to phase, and some of the organic phase was used as reflux with the remainder of the organic phase being returned to the extractor as the solvent stream. The organic phase was 1.9% by weight acrylic acid, 1.38% by weight acetic acid, 85.32% by weight ethyl acrylate, 2.1% by weight water, and 9.29% by weight toluene. The overhead aqueous phase was comprised of 1.14% by weight acrylic acid, 3.97% by weight acetic acid, 1.91% by weight ethyl acrylate, 92.98% by weight water, and 0.005% by weight toluene. The distillation residue composition was 99.34% by weight acrylic acid, 0.41% by weight acetic acid, 0.014% by weight ethyl acrylate, and 0.051% by weight water.

### Comparative Example R

An aqueous stream composed of 34.99% by weight acrylic acid, 2.5% by weight acetic acid, and 62.44% by weight water is fed to the top of a counter-current extractor at a rate of 4.8 g/min and contacted with a solvent composed of 0.438% by weight acrylic acid, 1.05% by weight acetic acid, 96.7% by weight ethyl acrylate, and 1.8% by weight water, entering at the bottom of the extractor at a rate of 3.03 g/min. The extraction was performed with approximately 6 theoretical stages. The aqueous raffinate contained 0.71% by weight acrylic acid, 1.56% by weight acetic acid, 2.09% by weight ethyl acrylate, and 95.64% by weight water. The organic extract, composed of 27.85% by weight acrylic acid, 1.65% by weight acetic acid, 57.97% by weight ethyl acrylate, and 12.47% by weight water was fed to a 20-tray one inch diameter Oldershaw distillation column at a rate of 5.07 g/min. The pressure at the top of the column was maintained at 165 mm Hg, the reflux rate at 2.0 ml/min, and the bottom temperature at 100° C. The condensed overhead was allowed to phase, and some of the organic phase was used as reflux with the remainder of the organic phase being returned to the extractor as the solvent stream. The organic phase was 0.438% by weight acrylic acid, 1.05% by weight acetic acid, 96.7% by weight ethyl acrylate, and 1.8% by weight water. The overhead aqueous phase was comprised of 0.75% by weight acrylic acid, 2.14% by weight acetic acid, 2.09% by weight ethyl acrylate, and 95.02% by weight water. The distillation residue composition was 96.53% by weight acrylic acid, 2.94% by weight acetic acid, 0.32% by weight ethyl acrylate, and 0.056% by weight water.

The data from Example 21 and Comparative Example R are further summarized in Table 6 for the various streams (Reference FIG. 1), whereas mass balances for these examples appear in Tables 7 and 8, respectively.

TABLE 6
Purification of Acrylic Acid with Ethyl Acrylate and Ethyl Acrylate/Toluene Mixed Solvent

| | Composition at | | | | | | | | | |
|---|---|---|---|---|---|---|---|---|---|---|
| | 10 | | 14 | | 16 | | 20 | | 32 | |
| | Extractor Feed | | Solvent | | Aq Raffinate | | Org Extract | | Dist Aq | |
| | EA/Tol | EtAcA | EA/Tol | EtAcA | EA/Tol | EtAcA | EA/Tol | EtAcA | EA/Tol | EtAcA |
| Comp | (% wt) | (% wt) | (% wt) | (% wt) | (% wt) | (% wt) | (% wt) | (% wt) | (% wt) | (% wt) |
| HAcA | 34.99 | 34.99 | 27.38 | 27.85 | 2.5 | 0.71 | 27.38 | 27.85 | 1.14 | 0.75 |
| HOAc | 2.5 | 2.5 | 1.6 | 1.65 | 2.6 | 1.56 | 1.6 | 1.65 | 3.969 | 2.14 |
| EtAcA | | | 54.08 | 57.97 | 1.9 | 2.09 | 54.08 | 57.97 | 1.906 | 2.09 |
| H2O | 62.44 | 62.44 | 10.7 | 12.473 | 92.99 | 95.64 | 10.7 | 12.473 | 92.98 | 95.02 |
| Tol | | | 6.2 | | 0.004 | | 6.2 | | 0.005 | |
| Flow | 5.2 | 4.8 | 6.4 | 6.05 | 2.77 | 2.45 | 6.2 | 5.07 | 0.62 | 0.60 |
| (g/min) | | | | | | | | | | |

| | Composition at | | | | | | | |
|---|---|---|---|---|---|---|---|---|
| | 34 | | 30 | | 36 | | 40 | |
| | Dist Res | | Reflux | | Dist Org | | Solvent makeup | |
| | EA/Tol | EtAcA | EA/Tol | EtAcA | EA/Tol | EtAcA | EA/Tol | EtAcA |
| Comp | (% wt) | (% wt) | (% wt) | (% wt) | (% wt) | (% wt) | (% wt) | (% wt) |
| HacA | 99.34* | 96.53* | 1.9 | 0.438 | 1.9 | 0.438 | | |
| HOAc | 0.41 | 2.94 | 1.38 | 1.05 | 1.38 | 1.05 | | |
| EtAcA | 0.014 | 0.32 | 85.32 | 96.7 | 85.32 | 96.7 | 0.9 | 1.0 |
| H2O | 0.051 | 0.056 | 2.1 | 1.8 | 2.1 | 1.8 | | |
| Tol | | | 9.29 | | 9.29 | | 0.1 | |
| Total | 1.6 | 1.44 | 4.2 | 3.2 | 3.98 | 3.03 | 0.05 | 0.67 |
| (g/min) | | | | | | | | |

*This value includes dimer

TABLE 7
Component Mass Balance for EA/Toluene Case, Example 21

| | Extractor Mass Balance | | | Column Mass Balance | | |
|---|---|---|---|---|---|---|
| | In | Out | Difference | In | Out | Difference |
| | (g/min) | (g/min) | (%) | (g/min) | (g/min) | (%) |
| HacA | 1.89548 | 1.82157 | 4.06% | 1.69756 | 1.67251 | 1.48% |
| HOAc | 0.18520 | 0.17442 | 6.18% | 0.09920 | 0.08631 | 12.99% |
| EtAcA | 3.41280 | 3.51375 | −2.87% | 3.35296 | 3.42484 | −2.14% |
| H20 | 3.33088 | 3.26062 | 2.15% | 0.66340 | 0.66129 | 0.32% |
| Tol | 0.37160 | 0.39691 | −6.38% | 0.38440 | 0.37163 | 3.32% |

$( \% ) Difference = \frac{( In - Out )}{In}$

TABLE 8
Component Mass Balance for Pure EtAcA Case,
Comparative Example R

| | Extractor Mass Balance | | | Column Mass Balance | | |
|---|---|---|---|---|---|---|
| | In | Out | Difference | In | Out | Difference |
| | (g/min) | (g/min) | (%) | (g/min) | (g/min) | (%) |
| HAcA | 1.6957 | 1.7023 | −0.39% | 1.412 | 1.4078 | 0.30% |
| HOAc | 0.1589 | 0.138 | 13.10% | 0.0837 | 0.087 | −3.99% |
| EtAcA | 3.5779 | 3.5584 | 0.55% | 2.9391 | 2.9472 | −0.27% |
| H20 | 3.0637 | 3.0978 | −1.11% | 0.6324 | 0.6255 | 1.09% |
| Tol | 0 | 0 | N/A | 0 | 0 | N/A |

$( \% ) Difference = \frac{( In - Out )}{In}$

What is claimed is:

1. A method of recovering acrylic acid from a mixture comprising acrylic acid, water and acetic acid comprising:
(a) extracting acrylic acid from said mixture with a solvent mixture comprising ethyl acrylate as the preponderant component thereof and an organic co-solvent selected from the group consisting of toluene, heptane, 1-heptene, methylcyclohexane, cycloheptane, cycloheptadiene, cycloheptatriene, 2,4-dimethyl-1,3 pentadiene, methylcyclohexene and methylenecyclohexene to form an extracted composition; and
(b) azeotropically distilling said extracted composition to recover acrylic acid.

2. The method according to claim 1, wherein said steps of extracting acrylic acid and azeotropically distilling the extracted composition are carried out in a continuous process to form a residue stream the preponderant component of which is acrylic acid.

3. The method according to claim 2, wherein said residue stream is composed of at least 98 weight percent (wt %) acrylic acid.

4. The method according to claim 3, wherein said residue stream is composed of at least 99% acetic acid.

5. The method according to claim 2, wherein said residue stream contains less than about 0.75 wt % acetic acid.

6. The method according to claim 5, wherein said residue stream contains less than about 0.5 wt % acetic acid.

7. The method according to claim 2, wherein said residue stream contains less than about 0.5 wt % water.

8. The method according to claim 7, wherein said residue stream contains less than about 0.1 wt % water.

9. The method according to claim 1, wherein the extracted composition comprises at least about 50 wt % ethyl acrylate.

10. The method according to claim 9, wherein the extracted composition contains at least about 20 wt % acrylic acid.

11. The method according to claim 1, wherein said organic co-solvent is toluene.

12. The method according to claim 1, wherein the weight ratio of ethyl acrylate to said organic co-solvent in said solvent mixture is from about 80:20 to about 95:5.

13. The method according to claim 12, wherein the weight ratio of ethyl acrylate to said organic co-solvent in said solvent mixture is from about 85:15 to about 95:5.

14. The method according to claim 13, wherein said organic co-solvent is toluene.

15. The method according to claim 1, wherein said process is operative to remove at least about 75 wt % of the acetic acid present in the mixture of acrylic acid, water and acetic acid undergoing purification.

16. The method according to claim 15, wherein said process is operative to remove at least about 80 wt % of the acetic acid present in the mixture of acrylic acid, water and acetic acid undergoing purification.

17. A method of recovering acrylic acid comprising:
(a) providing a feed stream containing acrylic acid, water, acetic acid, ethyl acrylate and an organic co-solvent selected from the group consisting of toluene, heptane, 1-heptene, methylcyclohexane, cycloheptane, cycloheptadiene, cycloheptatriene, 2,4-dimethyl-1,3 pentadiene, methylcyclohexene and methylenecyclohexene to a distillation column, wherein the weight ratio of ethyl acrylate to said organic co-solvent is from about 80:20 to about 95:5; and
(b) azeotropically distilling said feed stream to provide a residue stream, the preponderant component of which is acrylic acid.

18. The method according to claim 17, wherein said residue stream contains at least about 98 wt % acrylic acid.

19. The method according to claim 18, wherein said residue stream contains at least about 99 wt % acrylic acid.

20. The method according to claim 17, wherein said feed stream contains from about 5 to about 40 wt % water, from about 1 to about 4 wt % acetic acid and up to about 80 wt % acrylic acid.

21. The method according to claim 20, wherein said residue stream contains less than about 0.75 wt % acetic acid.

22. The method according to claim 21, wherein said residue stream contains less than about 0.5 wt % acetic acid.

23. The method according to claim 20, wherein said residue stream contains less than about 0.5 wt % water.

24. The method according to claim 23, wherein said residue stream contains less than about 0.1 wt % water.

25. The method according to claim 17, wherein said organic co-solvent is toluene.

26. The method according to claim 25, wherein the weight ratio of ethyl acrylate to toluene in said feed stream is from about 85:15 to about 95:5.

27. The method according to claim 17, wherein said process is operative to remove at least about 75 wt % of the acetic acid present in the feed stream undergoing purification.

28. The method according to claim 27, wherein said process is operative to remove at least about 80 wt % of the acetic acid present in said feed stream.

29. The method according to claim 17, wherein said azeotropic distillation is carried out with a temperature of about 100° C. about the lower portion of said distillation column.

30. The method according to claim 29, wherein the temperature about the central portion of said distillation column is maintained at a temperature of about 60° C. when azeotropically distilling said feed stream.
